(19) United States
(12) Patent Application Publication
(10) Pub. No.: US 2022/0313489 A1
(43) Pub. Date: Oct. 6, 2022

(54) SYSTEMS AND METHOD FOR SMART PHACO IN SURGICAL SYSTEMS

(71) Applicant: JOHNSON & JOHNSON SURGICAL VISION, INC., Irvine, CA (US)

(72) Inventors: Abraham Hajishah, Irvine, CA (US); Edith Fung, Diamond Bar, CA (US); Deep Mehta, Irvine, CA (US)

(21) Appl. No.: 17/634,426

(22) PCT Filed: Aug. 12, 2020
(86) PCT No.: PCT/IB2020/057591

§ 371 Date: Feb. 10, 2022

Related U.S. Application Data
(60) Provisional application No. 62/888,296, filed on Aug. 16, 2019.

(51) Int. Cl. A61F 9/007

(52) U.S. Cl. CPC A61F 9/00736

(57) ABSTRACT

A smart automated phacoemulsification surgical system for reducing variables significantly by introducing a relationship among related variables that share the same functionalities or where they depend on each other. The system optimizes power applied and and when the power reaches its maximum targeted value while the occlusion persists for an extended period of time. An automatic venting subsystem is configured to release cataract particles from the tip of the surgical instrument. An ultrasound power setting is configured to be reset to overcome power and occlusion stagnation in order to prevent applying too much ultrasound power and harming the eye tissues of a patient.

### CROSS REFERENCE TO RELATED APPLICATIONS

This application claims priority to U.S. Provisional Patent Application No. 62/888,296, filed on Aug. 16, 2019, the entire contents of which are hereby incorporated by reference.

### BACKGROUND

### Field of Invention

The present disclosure relates to auto phacoemulsification for ophthalmic surgery and, more specifically, the optimization of applied power during occlusion breaks to clear particles in a surgical handpiece tip.

### Description of Related Art

During ophthalmic surgery, an ophthalmic surgical apparatus is used to perform surgical procedures in a patient's eye. An ophthalmic surgical apparatus typically includes a handheld medical implement or tool, such as a handpiece with a tip and/or sleeve, and operating controls for regulating settings or functions of the apparatus and tool. Operation of the tool requires control of various operating settings or functions based on the type of tool used. Such apparatuses typically include a control module, power supply, an irrigation source, one or more aspiration pumps, as well as associated electronic hardware and software for operating a multifunction handheld surgical tool. The handpiece may include a needle or tip which is ultrasonically driven once placed within the incision to, for example, emulsify the lens of the eye. In various surgical procedures, these components work together in order to, for example, emulsify eye tissue, irrigate the eye with a saline solution, and aspirate the emulsified lens from the eye.

Intraocular pressure (IOP) is the fluid pressure inside the anterior chamber of the eye. In a normal eye, intraocular pressure may vary depending on the time of day, activities of the patient, fluid intake, medications, etc. Intraocular pressure may be measured as static (a specific value) or dynamic (a range of values). As can be appreciated, the static IOP and dynamic IOP of a patient's eye can fluctuate greatly during an ophthalmic surgery procedure. It is well-known that the IOP in an anterior chamber of the eye is required to be controlled and maintained during such surgical procedures in order to avoid damage to the patient's eye. For the correct function of the eye and its structure (e.g. shape) and to preserve sharp and undamaged vision, it is very important to keep the IOP in normal, physiological values.

An exemplary type of ophthalmic surgery is phacoemulsification. Phacoemulsification includes making a corneal and/or scleral incision and the insertion of a phacoemulsification handpiece that includes a needle or tip that is ultrasonically driven to emulsify, or liquefy, the lens. A phacoemulsification system typically includes a handpiece coupled to an irrigation source and an aspiration pump. The handpiece includes a distal tip that emits ultrasonic energy to emulsify a crystalline lens within the patient's eye. The handpiece includes one or more irrigation ports proximal to the distal tip and coupled to the irrigation source via an irrigation input line. The handpiece further includes an aspiration port at the distal tip that is coupled to the aspiration pump via an aspiration output line. Concomitantly with the emulsification, fluid from the irrigation source (which may be a bottle or bag of saline solution that is elevated above the patient's eye, to establish positive pressure by gravity, and/or with external pressure source) is irrigated into the eye via the irrigation line and the irrigation port(s). This fluid is directed to the crystalline lens in the patient's eye in order to maintain the anterior chamber and capsular bag and replenish the fluid aspirated away with the emulsified crystalline lens material. The irrigation fluid in the patient's eye and the crystalline lens material is aspirated or removed from the eye by the aspiration pump and line via the aspiration port.

Similarly, cataract surgery is a complex procedure performed by highly skilled surgeons using extremely complex and expensive equipment. The surgeon undergoes years of training to perfect their technique while using only a fraction of the system's capabilities and features. For example, cataract tissue, which may be denser, may be removed by aspiration. When the material has been emulsified or softened to the point where aspiration is sufficient to remove the material an occlusion break occurs. It is well known that excessive energy application after an occlusion break occurs, known as a post occlusion surge, could potentially damage the tissue. In practice, the surgeon may anticipate this occurrence and discontinue ultrasonic power to prevent any damage to the eye. If the occlusion break occurs faster than the surgeon can discontinue power, the surgeon may apply more power than needed. Studies have shown that the human reaction time is approximately 350 milliseconds (ms). That means the patient may be subjected to an additional 350 ms or more of ultrasonic energy every occlusion break.

For example, during segment removal, the surgeon may confront a multitude of decisions as he/she attempts to balance the inflow and outflow of fluid in the eye while trying to control the movement of material with the handpiece and deciding when to apply ultrasonic power. Additionally, lens material may create a blockage at the tip preventing fluid from being evacuated. This blockage can result in post-occlusion surge and lead to eye trauma. When faced with a potential post occlusion surge situation, the surgeon has to decide whether to preempt the surge by clearing the occlusion by applying power to knock the piece off the tip and having to reacquire the piece or discontinue the procedure by gradually (or quickly) releasing the foot pedal to change the pump speed and/or vacuum. Depending on the density of the material, length of occlusion, maximum aspiration rate, maximum vacuum and a wide variety of other factors, the occlusion may clear before the surgeon can take action. A disadvantage in releasing the footpedal is the fact that cataract lens material in the aspirating phacoemulsification handpiece may flow back into the eye chamber leading to a longer, less efficient cataract extraction.

Techniques to overcome post inclusion surge have been developed that include smaller or specialized tips that allow fluid to enter through a secondary port to allow continuous fluid flow. Alternatively, other techniques include modifying predefined vacuum or aspiration settings, adjusting vacuum manually during the procedure or automatically “on-the-fly”, and releasing the foot pedal to discontinue aspiration. These techniques have had varying levels of success.

Other techniques include steps to ramp from a user-defined baseline phaco energy setting on occlusion onset until the occlusion has cleared. The user-defined baseline setting, if too low, may cause extra phaco time due to the time required to ramp to an occlusion break power. On the other hand, if the user-defined baseline is too high, an excess of phaco energy can, and will, be imparted to the patient's eye.

Current Phaco algorithms involve complex settings and configurations during the cataract procedure and its implementation. Current implementations rely on surgeon experience to play with the foot pedal to remove the power and occlusion stagnation. What is needed is an auto solution for venting and power reset during occlusion.

### SUMMARY

The present invention provides a system for reducing variables significantly by introducing a relationship among related variables that share the same functionalities or where they depend on each other. Further, the system optimizes the power applied and when the power reaches its maximum targeted value while the occlusion persists for an extended period of time. The system triggers an automatic venting subsystem which releases cataract particles from the tip of the surgical instrument. Further, the system is configured to reset an ultrasound power setting to overcome the power and occlusion stagnation in order to prevent applying too much ultrasound power and harming the eye tissues of a patient. The disclosed smart auto phaco systems and methods reduces the number of variables by 50% and simplifies the user setting of auto phaco parameters.

### BRIEF DESCRIPTION OF THE DRAWINGS

The organization and manner of the structure and function of the disclosure, together with the further objects and advantages thereof, may be understood by reference to the following description taken in connection with the accompanying drawings, and in which:

FIG. 1 illustrates a diagram of an exemplary phacoemulsification/diathermy/vitrectomy system in accordance with the present disclosure, the system including a control module to control various features of the system;

FIG. 2 illustrates an alternative phacoemulsification/diathermy/vitrectomy system and illustrated connected to various components of the system in order to determine characteristics or features of the components;

FIG. 3 illustrates an auto phaco solution in accordance with the known art; and

FIG. 4 illustrates a smart auto phaco solution in accordance with at least one embodiment of the disclosed invention.

### DETAILED DESCRIPTION

The following description and the drawings illustrate specific embodiments sufficiently to enable those skilled in the art to practice the described system and method. Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

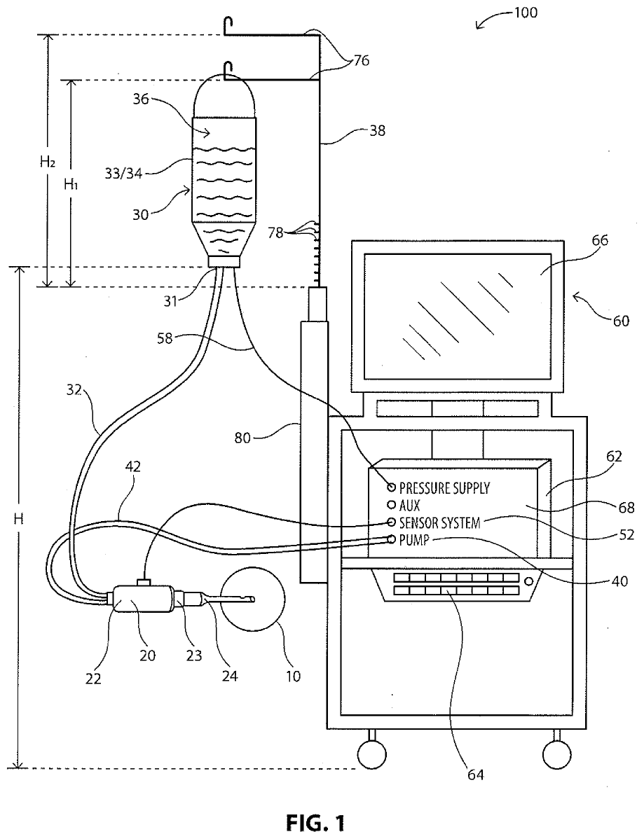

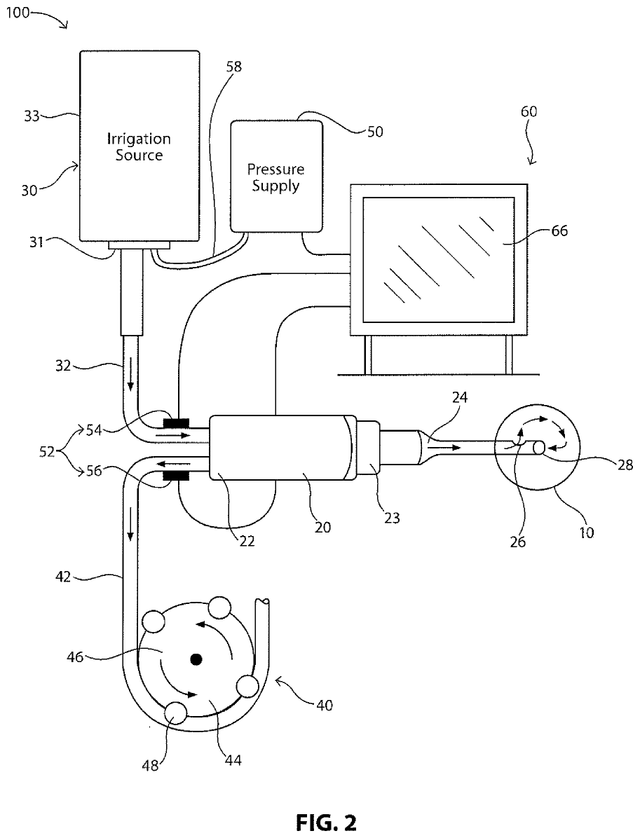

FIGS. 1 and 2 illustrate an exemplary phacoemulsification/diathermy/vitrectomy system 100. As illustrated, the system 100 includes, for example, a handpiece or wand 20, an irrigation source 30, an aspiration source 40, an optional pressure supply 50, and a control module 60. In illustrative embodiments, fluid is controllably directed through the system 100 in order to irrigate a patient's eye, illustrated representatively at 10, during an ocular surgical procedure. Various embodiments of the handpiece 20, irrigation source 30, aspiration source 40, optional pressure supply 50 and control module 60 are well known in the art and are embodied in this disclosure.

As illustrated in FIGS. 1 and 2, the irrigation source 30 is configured to supply a predetermined amount of fluid to the handpiece 20 for use during a surgical operation. Such fluid is supplied in order to, for example, stabilize or maintain a certain intraocular pressure (IOP) in the anterior chamber of the eye during surgery, as well as provide means for fluidly transporting any particles (e.g. lens particulates that are created during emulsification) out of the eye. Various aspects (e.g. the flow rate, pressure) of fluid flow into and out of the anterior chamber of the eye will typically affect the operations of the surgical procedure.

In illustrative embodiments, fluid may flow from the irrigation source 30 to the handpiece 20 via an irrigation line 32. The irrigation source 30 may be any type of irrigation source 30 that can create and control a constant fluid flow. In illustrative embodiments, the irrigation source is elevated to a predetermined height via an extension arm 38. In illustrative embodiments, the irrigation source 30 may be configured to be an elevated drip bag 33/34 that supplies a steady state of fluid 36 to the irrigation line 32. The pressure supply 50 may be coupled to the irrigation source 30 in order to maintain a constant pressure in the irrigation source 30 as fluid exits the irrigation source 30, as is known in the industry. Other embodiments of a uniform irrigation source are well known in the art.

During the surgical procedure, it is typically necessary to remove or aspirate fluid and other material from the eye. Accordingly, fluid may be aspirated from the patient's eye, illustrated representatively at 10, via the handpiece 20 to flow through an aspiration line 42 to the aspiration source 40. The aspiration source 40 may be any type of aspiration source 40 that aspirates fluid and material from the eye. In illustrative embodiments, the aspiration source 40 may be configured to be a flow-based pump 44 (such as a peristaltic pump) and/or a vacuum-based pump (such as a Venturi pump) that are well known in the art. The aspiration source 40 may create a vacuum system to pump fluid and/or material out of the eye via the aspiration line 42. A sensor system 52 may be present to measure the pressure that the vacuum creates. Other embodiments of an aspiration source are well known in the art.

The irrigation port 26 is fluidly coupled to the irrigation line 32 to receive fluid flow from the irrigation source 30, and the aspiration port 28 is fluidly coupled to the aspiration line 42 to receive fluid and/or material flow from the eye. The pressure in the aspiration line may be measured by the sensor system 52. The handpiece 20 and the tip 24 may further emit ultrasonic energy into the patient's eye, for instance, to emulsify or break apart the crystalline lens within the patient's eye. Such emulsification may be accomplished by any known methods in the industry, such as, for example, a vibrating unit (not shown) that is configured to ultrasonically vibrate and/or cut the lens, as is known in the art. Other forms of emulsification, such as a laser, are well known in the art. Concomitantly with the emulsification, fluid from the irrigation source 30 is irrigated into the eye via the irrigation line 32 and the irrigation port 26. During and after such emulsification, the irrigation fluid and emulsified crystalline lens material are aspirated from the eye by the aspiration source 40 via the aspiration port 28 and the aspiration line 42. Other medical techniques for removing a crystalline lens also typically include irrigating the eye and aspirating lens parts and other liquids. Additionally, other procedures may include irrigating the eye and aspirating the irrigating fluid within concomitant destruction, alternation or removal of the lens.

The aspiration source 40 is configured to aspirate or remove fluid and other materials from the eye in a steady, uniform flow rate. Various means for steady, uniform aspiration are well-known in the art. In illustrative embodiments, the aspiration source 40 may be a Venturi pump, a peristaltic pump, or a combined Venturi and peristaltic pump. In illustrative embodiments, and as shown in FIG. 2, a peristaltic pump 44 may be configured to include a rotating pump head 46 having rollers 48. The aspiration line 42 is configured to engage with the rotating pump head 46 as it rotates about an axis. As the pump head 46 rotates the rollers 48 press against the aspiration line 42 causing fluid to flow within the aspiration line 42 in a direction of the movement for the rollers 48. Accordingly, the pump 44 directly controls the volume or rate of fluid flow, and the rate of fluid flow can be easily adjusted by adjusting the rotational speed of the pump head 46. Other means of uniformly controlling fluid flow in an aspiration source 40 are well known in the art. When the aspiration source 40 includes a combined Venturi and peristaltic pump, the aspiration source 40 may be controlled to automatically switch between the two types of pumps or user controlled to switch between the two types of pumps.

In illustrative embodiments, the control module 60 is configured to monitor and control various components of the system 100. For instance, the control module 60 may monitor, control, and provide power to the pressure supply 50, the aspiration source 40, and/or the handpiece 20. The control module 60 may be in a variety of forms as known in the art. In illustrative embodiments, the control module 60 may include a microprocessor computer 62, a keyboard 64, and a display or screen 66, as illustrated in FIGS. 1 and 2. The microprocessor computer 62 may be operably connected to and control the various other elements of the system, while the keyboard 64 and display 66 permit a user to interact with and control the system components as well. In an embodiment a virtual keyboard on display 66 may be used instead of keyboard 64. A system bus 68 may be further provided to enable the various elements to be operable in communication with each other. The control module 60 may be powered by an energy source. One skilled in the art would appreciate that the energy source may be a power source—such as a 110v plug—or conventional commercial power sources.

The screen 66 may display various measurements, criteria or settings of the system 100—such as the type of procedure, the phase of the procedure and duration of the phase, various parameters such as vacuum, flow rate, power, and values that may be input by the user, such as bottle height, sleeve size, tube length (irrigation and aspiration), tip size, vacuum rate. The screen 66 may be in the form of a graphical user interface (GUI) 70 associated with the control module 60 and utilizing a touchscreen interface, for example. The GUI 70 may allow a user to monitor the characteristics of the system 100 or select settings or criteria for various components of the system. For instance, the GUI 70 may permit a user to select or alter the maximum pressure being supplied by the pressure supply 50 to the irrigation source 30 via line 58. The user may further control the operation of the phase of the procedure, the units of measurement used by the system 100, or the height of the irrigation source 30, as discussed below. The GUI 70 may further allow for the calibration and priming of the pressure in the irrigation source 30.

In illustrative embodiments, the system 100 may include a sensor system 52 configured in a variety of ways or located in various locations. For example, the sensor system 52 may include at least a first sensor or strain gauge 54 located along the irrigation line 32 and a second sensor or strain gauge 56 located along the aspiration line 42, as illustrated in FIG. 2. Other locations for the sensors 54 and 56 are envisioned anywhere in the system 100, e.g. on the handpiece 20, and may be configured to determine a variety of variables that may be used to determine pressure measurements in the aspiration line, as discussed below. This information may be relayed from the sensor system 52 to the control module 60 to be used in the determination of the presence of an occlusion break. The sensor system 52 may also include sensors to detect other aspects of the components used in the system, e.g. type of pump used, type of sleeve used, gauge of needle tip (size), etc.

Those of skill in the art will recognize that any step of a method described in connection with an embodiment may be interchanged with another step without departing from the scope of the invention. Those of skill in the art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed using a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Any options available for a particular medical device system may be employed with the present invention. For example, with a phacoemulsification system the available settings may include, but are not limited to, irrigation, aspiration, vacuum level, flow rate, pump type (flow based and/or vacuum based), pump speed, ultrasonic power (type and duration, e.g. burst, pulse, duty cycle, etc.), irrigation source height adjustment, linear control of settings, proportional control of settings, panel control of settings, and type (or “shape”) of response.

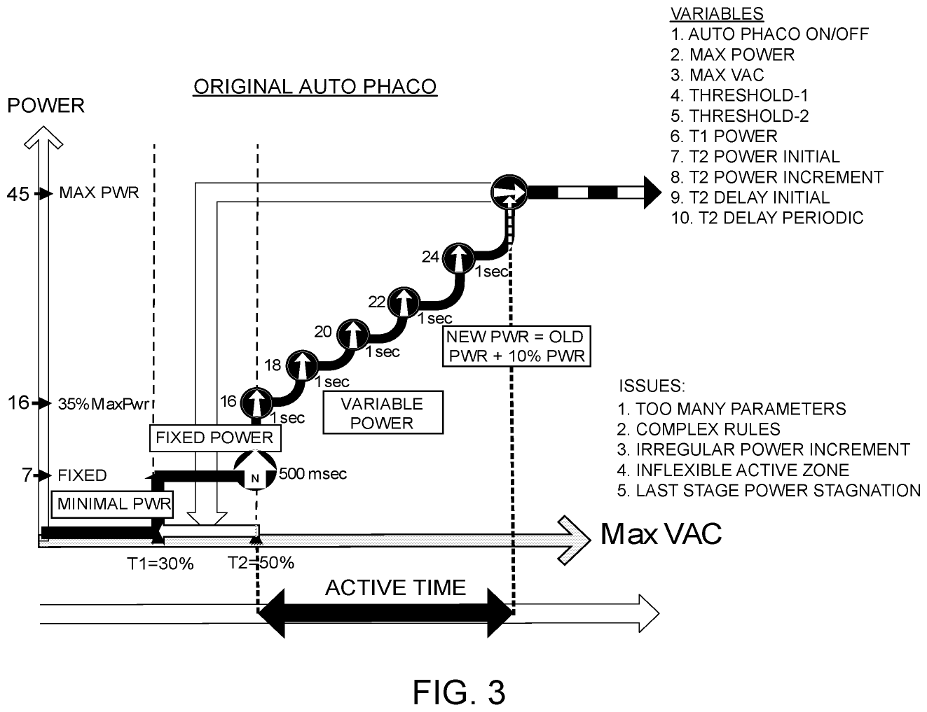

FIG. 3 illustrates key data points of an original auto phaco system. Issues related to the original auto phaco system include, but are not limited to, too many parameters, complex rules, irregular power increment, inflexible active zone, and last stage power stagnation.

The disclosed systems and methods provide for smart phaco in surgical systems by simplifying current Auto Phaco procedures and improve its performance. The number of variables is reduced by 50% and the amount of required user settings is thereby simplified. In addition, the system may provide a capability to auto reset/vent in the event of an extended occluded tip causing the maximum applied power to stagnate and not be effective at breaking up the particles at the surgical tip. Max power stagnation may be harmful to a patient.

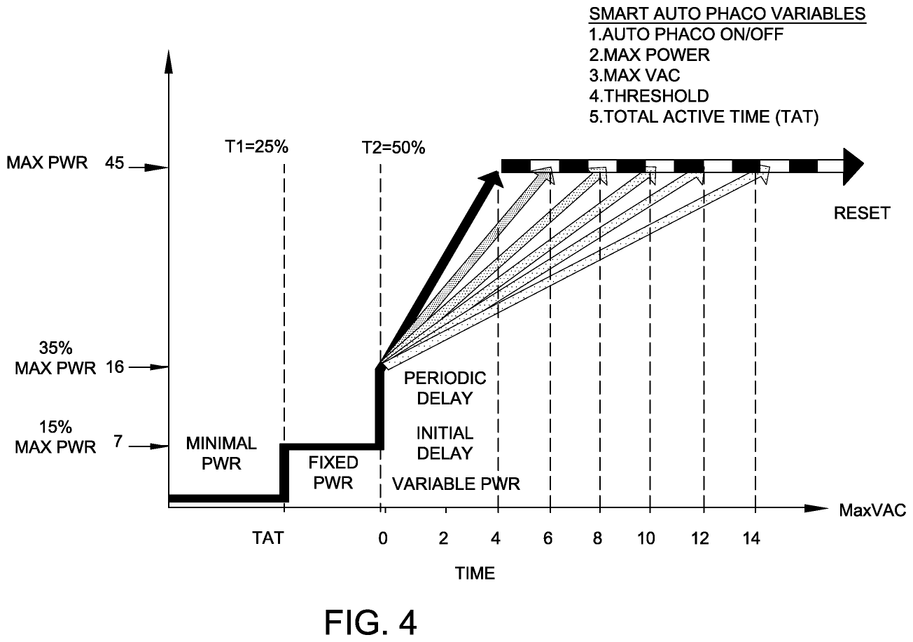

FIG. 4 illustrates key data points of a smart auto phaco system in accordance with one or more embodiments of the disclosed invention. In a first embodiment, variables may be reduced significantly. This may be accomplished by introducing a relationship among related variables that share the same functionalities or where they depend on each other. In addition, the system may optimize power applied and when the power reaches its maximum targeted value while the occlusion persists for an extended time. The system may trigger an automatic venting subsystem that is configured to release cataract particles from the surgical tip. In addition, the system may reset an ultrasound power setting to overcome the power and occlusion stagnation in order to prevent applying too much ultrasound power and harming the eye tissues of the patient.

In one non-limiting example, the original Auto Phaco would require a system user, such as a surgeon or surgical assistant, to select 10 variables. In this example, the Smart Auto Phaco system would only require 5 variables to be selected and the next 5 variables would be extracted and calculated based on the first 5 selections. This removes complexity and indecision about variable selection.

The following is a comparison of variables:

| Auto Phaco variables | Smart Auto Phaco Variables |
|---|---|
| 1—Auto Phaco ON/OFF | 1—Auto Phaco ON/OFF |
| 2—Max Power | 2—Max Power |
| 3—Max VAC | 3—Max VAC |
| 4—Threshold-1 | 4—Vacuum Threshold |
| 5—Threshold-2 | 5—TAT (Total Active Time) |
| 6—Thresh-1 Power | |
| 7—Thresh-2 Initial Power | |
| 8—Thresh-2 Power Increment | |
| 9—Thresh-2 Initial Delay | |
| 10—Thresh-2 Periodic Delay | |

The following formulas calculate the rest of variables for Smart Auto Phaco
Threshold-1 = ½ Threshold
Threshold-2 = Threshold
Initial Power = 35% of Max Power
Power Increment (PI) = 2% (fixed value)
Periodic Delay = PI * TAT/(65% * Max Power)
Initial Delay = ½ Periodic Delay

| Auto Phaco selected variables | |
|---|---|
| variables | Smart Auto Phaco calculated |
| Threshold-1 | ½ Threshold |
| Threshold-2 | Threshold |
| Thresh-1 Power | 15% of Max Power |
| Thresh-2 Power Initial | 35% of Max Power |
| Thresh-2 Power Increment | Fixed 2% |
| Thresh-2 Initial Delay | Calculated based on TAT |
| Thresh-2 Periodic Delay | Calculated based on TAT |

In an embodiment of the present invention, selected variables may be used for calculated variables. For example, selected variables may include a Max Power of 45%, a Max Vacuum of 500 mmHg, a chosen Threshold of 50% and a Total Active Time (TAT) of 14 seconds. Such selected variables may provide for calculated thresholds, such as: Threshold-1=(½)*Threshold*Max Vacuum=125 mmHg; Threshold-1=Threshold*Max Vacuum=250 mmHg; Threshold-1 Power=15%*Max Power=7%; Initial Power=35%*Max Power=16%. Additional selected variables may include a fixed Power Increment of 2%, a Periodic Delay equal to about 957 ms and an Initial Delay equal to about 479 ms.

In an embodiment of the present invention, selected variables may be used for calculated variables. For example, selected variables may include a Max Power of 55%, a Max Vacuum of 500 mmHg, a chosen Threshold of 60% and a TAT (total active time) of 6 seconds. Such selected variables may provide for calculated thresholds, such as: Threshold-1=(½)*Threshold*Max Vacuum=150 mmHg; Threshold-1=Threshold*Max Vacuum=300 mmHg; Threshold-1 Power=15%*Max Power=8%; Initial Power=35%*Max Power=19%. Additional selected variables may include a fixed Power Increment of 2%, a Periodic Delay equal to about 410 ms and an Initial Delay equal to about 205 ms.

In another non-limiting example within the scope and spirit of the disclosed invention, the vacuum of the surgical system may remain above Threshold 2 while the power reaches its max target value. In prior systems, a user would need to release the foot pedal to reset the power and activate venting. In the present invention, this action is automatic without releasing the foot pedal with a timer tracking time when the max targeted power is reached. The timer may be triggered and if such condition exceeds over 3 seconds, the venting and power reset are activated. This action may also release particles at the hand-piece and then grab them immediately at different angles much faster than when using a foot pedal for the same action.

It is understood that the above examples and variables are meant to be exemplary and non-limiting.

In yet another embodiment of the disclosed invention, the smart auto phaco system may be enabled to be a self-learning feature which can aggregate large datasets over time for a given surgeon and perform statistical analysis with the objective of minimizing ultrasound energy in the eye yet effectively emulsifying all grades of cataract particles automatically. The datasets may contain parameters including but not limited to surgeon specific datasets for all cases, such as Program settings (Phaco), aspiration flow, aspiration vacuum, foot pedal treadle position, ultrasound maximum power setting, ultrasound power modality, irrigation pressure and/or bottle height, and/or intraoperative IOP (predicted or actual). The datasets may contain parameters including but not limited to clinical specific datasets for all cases, such as cataract grade, patient's gender and age, patient's geographical location, patient's health history to include prescribed medications, ongoing disease and prescribed treatments, prior anterior and posterior surgeries, and/or patient's biological indicators such as lipid profile, blood glucose and A1C levels. The datasets may contain parameters including but not limited to environmental specific datasets for all cases, such as geographical location of the clinic, system and patient, average temperature, relative humidity, and/or particulate matter for specified geographic location.

In addition to ultrasound power, self-learning algorithm/feature may be expanded to automatically adjust aspiration flow rate, vacuum, and irrigation pressure for a given case that considers surgeon's technique and patient's biological and medical/health history. For example, the algorithm can learn how a given surgeon applies vacuum and ultrasound power based on historical foot pedal aspiration flow/vacuum datasets for patient with similar cataract grade, gender, age and medical history.

In addition to ultrasound power, self-learning algorithm/feature may be expanded to automatically adjust aspiration flow rate, vacuum and irrigation pressure for a given case that considers surgeon's technique and patient's biological and medical/health history.

The previous description is provided to enable any person skilled in the art to make or use the disclosed embodiments. Various modifications to these embodiments will be readily apparent to those skilled in the art, and the generic principles defined herein may be applied to other embodiments without departing from the spirit or scope of the invention. Thus, the present disclosure is not intended to be limited to the embodiments shown herein but is to be accorded the widest scope consistent with the principles and novel features disclosed herein.

What is claimed is:

1. A smart auto phaco phacoemulsification system, comprising:
a surgical console having at least one system bus communicatively connected to at least one computing processor capable of accessing at least one computing memory associated with the at least one computing processor, wherein the surgical console is configured to receive one or more configuration variables;
a surgical instrument having at least one surgical tip; and
a venting system of the surgical instrument configured to vent at least one or more particles within the at least one surgical tip in accordance with the one or more configuration variables.

2. The system of claim 1, wherein the one or more configuration variables include one or more of: auto phaco on/off, max power, max VAC, threshold, and total active time (TAT).

3. The system of claim 2, wherein the one or more variables are entered into the system by a user.

4. The system of claim 2, wherein the one or more variables are based on historical data gathered over time with respect to a specific surgeon.

5. The system of claim 2, wherein the one or more variables are based at least in part on user-entered variables and historical data.

6. The system of claim 2, wherein additional variables are determined in response to the one or more variables being related, sharing same functionalities, or depending on each other.

7. The system of claim 6, wherein the additional variables are extracted and calculated from the inputted one or more variables.

8. The system of claim 6, wherein the additional variables cause a power reset.

9. The system of claim 6, wherein the additional variables cause the venting system to automatically vent one or more particles from the surgical tip.

10. An auto phaco system including a surgical console and a surgical instrument, the system having at least one processor coupled to a memory, the auto phaco system configured to: receive one or more data inputs as initial data settings, extracting one or more further data inputs from the received one or more data points, and causing power reset and automatic venting of one or more particles within a tip of the surgical instrument in response to determined power settings.

11. The system of claim 10, wherein the received one or more data points are received from a user interface.

12. The system of claim 10, wherein the received one or more data points are based on historical data.

13. The system of claim 10, wherein the received one or more data points are received from a user interface and based on historical data.

14. The system of claim 10, further comprising a timer configured to track time when a max targeted power is reached during a phacoemulsification surgical procedure.

15. The system of claim 14, wherein a power reset is commenced in response to the timer reaching a certain value.

16. The system of claim 15, wherein the certain value is 3 seconds.

17. The system of claim 14, wherein a venting subsystem of the surgical instrument is activated in response to the timer reaching a certain value.

18. The system of claim 17, wherein the certain value is 3 seconds.

19. The system of claim 17, wherein activation of the venting subsystem causes one or more particles within a tip of the surgical instrument are vented.
